(19) United States
(12) Patent Application Publication
(10) Pub. No.: US 2005/0118713 A1
(43) Pub. Date: Jun. 2, 2005

(54) Morula derived embryonic stem cells

(76) Inventors: Nikolai Strelchenko, Deforest, WI (US); Yury Verlinsky, Chicago, IL (US)

Correspondence Address: WALLENSTEIN WAGNER & ROCKEY, LTD, 311 SOUTH WACKER DRIVE, 53RD FLOOR, CHICAGO, IL, 60606, US

(21) Appl. No.: 10/951,015

(22) Filed: Sep. 27, 2004

Related U.S. Application Data
(63) Continuation-in-part of application No. 10/436,306, filed on May 12, 2003.

(51) Int. Cl. C12N 5/08

(52) U.S. Cl. 435/366

(57) ABSTRACT

A method for isolating a human pluripotent embryonic stem cell line derived from culturing morula stage human embryo cells is disclosed. The method includes culturing the cells in close contact with a feeder cell layer to inhibit differentiation of the cells. A preparation of human pluripotent embryonic stem cells derived from culturing morula stage human embryo cells is also disclosed.

### CROSS-REFERENCE TO RELATED APPLICATIONS

The present application is a continuation-in-part of U.S. application Ser. No. 10/436,306 filed May 12, 2003, which is expressly incorporated herein by reference.

### FIELD OF THE INVENTION

The present invention generally relates to establishing embryonic stem cells. More specifically, the present invention relates to a method of isolating human embryonic stem (ES) cells derived from human morula stage embryos creating stem cell lines for use in cell therapy.

### BACKGROUND OF THE INVENTION

Currently established human ES cell lines are derived from the inner cell mass of a human blastocyst. The blastocyst is the first stage of embryo differentiation. Typically, day-5 blastocysts are used to derive ES cell cultures. A normal day-5 human embryo in vitro consists of between 200 to 250 cells. A majority of these cells contribute to the trophectoderm. In order to derive ES cell cultures, the trophectoderm is removed, either by microsurgery or immunosurgery (antibodies used to free the inner cell mass). At this stage of development, the inner cell mass is composed of between 30 to 34 cells. (Bongso, A Handbook on Blastocyst Culture, Singpore: 1999).

By way of background, after a human oocyte is fertilized in vitro by a sperm cell, the following events occur according to a fairly predictable time line. Day 1 is approximately 18-24 hours following in vitro fertilization or intracytoplasmic sperm injection. By Day 2, approximately 24-25 hours post fertilization, the zygote undergoes the first cleavage to produce a 2-cell embryo. By Day 3, the embryo reaches the 8-cell stage known as the morula, an early stage of embryo development characterized by equal and pluripotent blastomeres. During the morula stage, the genome of the embryo begins to control its own development. Any maternal influences from the presence of mRNA and proteins in the oocyte cytoplasm are significantly reduced. By Day 4, the cells of the embryo adhere tightly to each other through a process called compaction. By Day 5, the cavity of the blastocyst is complete and the inner cell mass begins to separate from the outer layer or trophectoderm that surrounds the blastocyst. This is the first observable sign of cell differentiation in the embryo.

An advantage of the use of blastomeres, or cells taken from the morula stage embryo, in the present invention, is that the blastomeres differ from the cells from the inner cell mass (ICM) of the blastocyst, both in size of the adjacent cytoplasm and gene pattern expression. Upon removal of the zona pellucida from the morula, all cells are pluripotent, meaning they retain the ability to produce a variety of differentiated cells. Morula derived ES cells have potential to be more pluripotent than ES cells established from the ICM of a blastocyst. The transcription factor Oct-4 is considered a marker for pluripotency of stem cells and is first detected in the nuclei of 8-16 cell morula, increasing in early blastocysts, and declines in late blastocysts, in which most Oct-4 protein is confined to the inner cell mass (ICM) region. (Liu, “Effect of Ploidy and Parentl Genome Composition on Expression of Oct-4 Protein in Mouse Embryos” Gene Expr. Patterns. 2004 July: 4(4): 433-41). Isolated prior to the onset of embryonic differentiation, morula derived ES cells tend to have less spontaneous differentiation, because they were isolated prior to first differentiation, whereas ES cells established from the ICM of blastocysts have already proceeded with differentiation. With the exception of humans, morula derived ES cells have been established in various other species, such as mouse, mink, and bovine. (Eistetter, “Pluripotent Embryonal Stem Cells can be Established from Disaggregated Mouse Morulae” Devel. Growth and Diff. 31, 275-282; Sukoyan, M. A.; Vatolin, S. Y.; Golubitsa, A. N.; Zhelezova, A. I.; Semenova, L. A.; Serov, O. L.; Embryonic Stem Cells Derivedfrom Morulae, Inner Cell Mass, and Blastocysts of Mink: Comparisons of their Pluripotencies, Mol. Reprod. Dev. 1993 October 36(2): 148-58; Stice, S. L.; Strelchenko, N. S.; Keefer, C. L.; Matthews, L.; Pluripotent Bovine Embryonic Stem Cell Lines Direct Embryonic Developments Following Nuclear Transfer, Biol Reprod. 1996 January; 54(1): 100-110; Strelchenko, N.; Stice, S.; WO 95/16770, Ungulate Preblastocyst Derived Embryonic Stem Cells and thereof to Produce Cloned Transgenic and Chimeric Ungulates,). The present invention is a method for isolating human morula derived ES cells, which are more pluripotent than cells derived from the blastocyst stage, making the present ES cell lines highly useful in cell therapy.

### SUMMARY OF THE INVENTION

The present invention is a method for isolating a human pluripotent embryonic stem cell line comprising the steps of: providing a morula stage human embryo cell; positioning the morula cells onto a feeder cell layer; culturing the morula cells to create multiple layers of cells; and, passaging the multiple layers of cells onto a second culturing medium for the proliferation of embryonic stem cells. In another embodiment, in the step of placing the morula cells onto the feeder cell layer, the morula cells are positioned in close contact with the feeder cell layer. In still another embodiment, the morula cells are positioned underneath the feeder cell layer.

Other features and advantages of the invention will be apparent from the following specification taken in conjunction with the following Figures.

### BRIEF DESCRIPTION OF THE DRAWINGS

FIG. 1 illustrates a 12-16 cell stage morula placed underneath a human fibroblasts feeder layer.

FIG. 2 illustrates a 12-16 cell stage morula placed underneath a mouse fibroblast feeder layer.

FIG. 3A illustrates the morphology of an ES cell colony derived from a morula stage embryo.

FIG. 3B illustrates the morphology of an ES cell colony derived from a blastocyst stage embryo.

FIG. 4 illustrates positive expression for alkaline phosphatase in a morula derived stem cell colony (purple color).

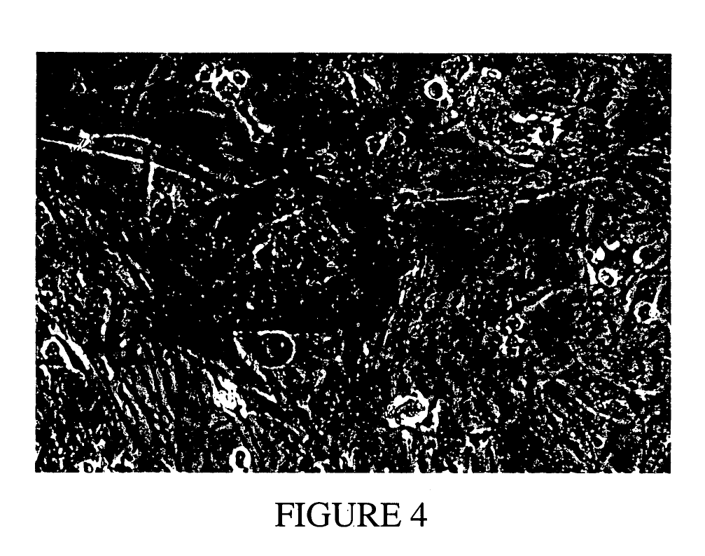

FIG. 5A illustrates a euploid karyotype female ES cell line in a morula derived ES cell.

FIG. 5B illustrates a euploid karyotype male ES cell line in a morula derived ES cell.

FIG. 6 illustrates detection of Oct-4 with RT-PCR products.

FIG. 7 illustrates localization of marker Tra-2-39 and Oct-4 in morula derived ES cells.(Tra-2-39 is green color and Oct-4 is red color).

### DETAILED DESCRIPTION

While this invention is susceptible of embodiments in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated.

The present invention is directed to a method of isolating human embryonic stem (ES) cells derived from morula stage human embryos. This includes embryos obtained after in vitro fertilization of allogeneic oocyte or after nuclear transfer of a human diploid cells into an enucleated allogeneic oocyte. In the preferred embodiment, the cells will be obtained from human morula stage embryos and are progenitors of the subject human embryonic stem cells. As an early or late stage morula embryo, the cells have not reached the blastocyst phase of development, and therefore remain equal and pluripotent.

The first step in the method of the present invention is to provide a morula stage human embryo cells. Morula stage human embryo cells are available through in vitro fertilization techniques that are known in the art and are available through Reproductive Genetics Institute (RGI). As the morula stage is prior to the blastocyst stage, it is important to determine at what stage the developing cells are in. There are a number of signs indicating the onset of the blastocyst stage of development, generally when cell count reaches between 20-32 cells in the embryo. Cells that have entered into the blastocyst stage are morphologically distinct from their morula stage precursors. The gene pattern expressions are also distinguishable. One indication is the presence of interferon tau (IFN-tau), an exclusive product released by the trophectoderm that functions as a fetal-maternal recognition mechanism. (Larson, M. A.; Kimura, K.; Kubisch, H. M.; Roberts, R. M.; Sexual Dimorphism Among Bovine Embryos in their Ability to make the Transition to Expanded Blastocyst and in the Expression of the Signaling Molecule IFN-tau. Proc. Natl. Acad. Sci. U.S.A. 2001 Aug. 14; 98(17):9677-82). The presence of interferon tau shows that the embryo is past the morula stage of development.

Another stage indicator is a drop in detectable MRNA estrogen receptor levels detectable at the one-cell, two-cell, and four-cell stage, but undetectable at the five- to eight-cell and morula stages. Upon reaching the blastocyst stage, the mRNA estrogen receptors become detectable again. (Ying, C.; Lin, D. H.; Estrogen-modulated Estrogen Receptor x Pit-1 Protein Complex Formation and Prolactin Gene Activation Require Novel Protein Synthesis, J. Biol. Chem. 2000 May 19; 275(20):15407-12). Other examples include: bovine embryos displaying high sensitivity to ouabain (potent inhibitor of the Na/K-ATPase), with enzyme activity undergoing a 9-fold increase from the morula stage to the blastocyst stage (Watson, A. J.; Barcroft, L. C.; Regulation of blastocyst formation, Front Biosci. 2001 May 1; 6:D708-30); mouse embryos showing different comparative mRNA expression patterns at the 2-cell, 4-cell, 8-cell morula, and blastocyst stages using a differential display (Lee, K. F.; Chow, J. F.; Xu, J. S.; Chan, S. T.; Ip, S. M.; Yeung, W. S.; A Comparative Study of Gene Expression in Murine Embryos Developed in vivo, Cultured in vitro, and Cocultured with Human Oviductal Cells using Messenger Ribonucleic Acid Differential Display, Biol Reprod. 2001 March;64(3):910-7); transition from morula stage to blastocyst stage of development was accompanied by a similar transformation of transcription Igf2 from biallelic to monoallelic (Ohno, M.; Aoki, N.; Sasaki, H.; Allele-specific detection of nascent transcripts by fluorescence in situ hybridization reveals temporal and culture-induced changes in Igf2 imprinting during pre-implantation mouse development, Genes Cells. 2001 March;6(3):249-59); serious changes in gene pattern expression displaying a distinctive but unstable maternal methylation pattern persisting during the morula stage, and disappearing in the blastocyst stage, where low levels of methylation are present on most DNA strands independently from parental origin (Hanel, M. L.; Wevrick, R.; The role of genomic imprinting in human developmental disorders: lessons from Prader-Willi syndrome, Clin. Genet. 2001 March; 59(3): 156-64.). These examples provide potential guidelines for determining between the two stages of embryo development—the morula cells from the blastocyst.

Cells from these two stages of development are morphologically different. Before morula stage cells differentiate into trophectoderm and the inner cell mass, aggregation of morula blastomeres occurs. This aggregation can be visually identified as the compact morula. An analogous cell compaction occurs in the inner cell mass prior to differentiation of cells into ectoderm, endoderm and mesoderm progenitor cells. To prevent further differentiation of the inner cell mass and to isolate embryonic stem cells out of the blastocyst, the inner cell mass is disaggregated and placed onto a cell feeder layer. A similar approach can be used for isolating morula derived embryonic stem cells, wherein the compact morula cells, or blastomeres are disaggregated.

In the present invention, culturing morula cells, or blastomeres, in a specific manner onto a feeder cell layer prevents differentiation. Experimental evidence supports a direct correlation between the efficiency of ES cell line generation and the contact quality between the feeder cell layer and the morula blastomeres. It has been shown that the contact between embryo cells, for example, bovine embryonic cells, and the feeder layer promotes proliferation, and established ES-cell lines. (Strelchenko, N.; Stice, S.; WO 95/16770, Ungulate Preblastocyst Derived Embryonic Stem Cells and thereof to Produce Cloned Transgenic and Chimeric Ungulates.)

The feeder cell layer can be of several types, including, allogeneic fibroblast feeder layer, xenogeneic fibroblast feeder layer, or cellular matrix. For example, it has been reported that using buffalo rat liver cells prevents the differentiation of mouse ES cells, through the production of leukemia inhibitor factor (LIF). (Smith, A. G.; Heath, J. K.; Donaldson, D. D.; Wong, G. G.; Moreau, J.; Stahl, M.; Rogers, D.; Inhibition of Pluripotential Embryonic Stem Cell Differentiation by Purified Polypeptides, Nature 1988 Dec. 15;336(6200):688-90). Other types of cells, such as cells from human placenta and fibroblasts could also be used as feeder layers producing other forms of differentiation inhibiting factors and proliferation of ES-cells. (Richards, M.; Fong, Chui-Yee; Chan, Woon-Khiong; Wong, Peng-Cheang; Bongso, Ariff: Human Feeders Support Prolonged Undifferentiated Growth of Human Inner Cell Masses and Embryonic Stem Cells, Nature Biotechnology September 2002, Vol. 20 933-936) (Miyamoto, Kanji; Hayashi, Kazuhiko; Suzuki, Toshio; Ichihara, Shinji; Yamada, Tomoaki; Kano, Yoshio; Yamabe, Toshio; Ito, Yoshihiro: Human Placenta Feeder Layers Support Undifferentiated Growth of Primate Embryonic Stem Cells, Stem Cells 2004; 22:433-440). Using the approach described herein, the cell layers that provide for the production of ES cell lines and ES colonies may be identified by routine screening to select for other cell layers.

In an alternate approach, the morula stage embryo can be cultured in a cell culture medium. The cell culture medium contains factors which inhibit differentiation and enable the isolation of ES cell lines and colonies. For example, the morula may be cultured in an LIF containing culture medium or any other factor containing culture medium, which prevents the differentiation of blastomeres. As one skilled in the art will appreciate, selection of the appropriate feeder cell layer or culture is not limited to the present examples.

Preferably, the individual morula or blastomere cells will be placed in contact with a fibroblast feeder layer. The feeder cell layers may be produced according to well-known methods. For example, mouse fibroblast feeder layers may be prepared in the following manner. Mouse fetuses are obtained during the 12-14 day of gestation period. The head, liver, heart, and alimentary tracts are removed. The remaining tissue is washed in phosphate buffered saline incubated at 37° C. in a solution of 0.05% trypsin 0.02%; EDTA. The mouse cells are placed in tissue culture flasks containing a culture medium that provides for the support of the feeder layer and the blastomeres.

While not limited, an example of a suitable culture medium comprises a modified Eagle's Medium containing non—essential amino acids (alanine, asparagine, aspartic acid, glutamic acid, glycine, proline and serine), ribonucleoside and 21 deoxyribonucleosides (hereinafter, MEM-Alpha) supplemented with 100 IU/ml penicillin, 50 μg/ml streptomycin, 10% fetal calf serum (FCS) and 0.1 mM 2-mercaptoethanol. The plated cells are cultured, preferably at 37° C., 4-5% C02 and 100% humidity until monolayers are produced. In alternate embodiments, one or more of these moieties may be non-essential to the growth of the blastomeres and generation of ES cells. For instance, the amount of FCS may be reduced to about 5% without detrimental growth effects.

After fibroblast cell monolayers are produced, the monolayer cells are treated. In one embodiment, the cells are treated with mitomycin C at a concentration of about 10 mg/ml for about three hours. Treatment by mitomycin C inhibits DNA synthesis, thus inhibiting cell division of the feeder layer cells, while concurrently providing for the monolayer cells to support the growth of co-cultured morula cells.

After formation of a suitable feeder cell layer or a cell culture medium, the blastomeres are cultured for a time sufficient to provide for the formation of embryonic stem cell colonies. In the preferred embodiment, the pre-blastocyst derived blastomeres are layered to be in contact with the fibroblast feeder layer. Providing significant cell-to-cell contact between the blastomeres and feeder layer generates ES cell lines more efficiently, and prevents differentiation of the morula blastomeres. Prevention of differentiation is theorized to be due to the membrane-associated differentiating inhibiting factors produced by the fibroblasts. Interestingly, based on visual observations blastomeres do not appear to go through an ICM stage as they multiply into ES cells. This may be another result of the cell-to-cell contact. In the absence of cell-to-cell contact, the pre-blastocyst derived blastomeres differentiate into trophoblast vesicles. Therefore, it is important to maximize the cell-to-cell contact.

In a preferred embodiment, the morula or blastomeres are placed underneath the feeder layer. In another embodiment, ES cell lines can be isolateded when the blastomeres are placed on top of the feeder layer. In yet another embodiment, it may be possible to sandwich the morula or blastomeres between two feeder cell layers, or placing the morula cells onto a cellular matrix and its derivation. In any of these embodiments, maximizing cell-to-cell contact appears to be the key to preventing differentiation.

Once the blastomeres have been cultured for a sufficient period of time, generally on the order of seven to ten days post initiation of culturing, the cells must be passaged. The cells should be passaged when they begin to exhibit an embryoid-like appearance, thus indicating the onset of cell differentiation. However, other factors will effect the timing for passaging, such as, the particular feeder cell layer type, the orientation of the cells on the feeder cell layer, the stage of the pre-blastocyst blastomeres, and the composition of the culture medium. The cells must be passaged to another feeder cell layer or a culture medium which prevents differentiation and provides for the growth of ES cells.

Preferably, passage will be effected without chemicals or proteases such as trypsin, which may be traumatic to the ES cells. For example, trypsin may denature ES protein and cell receptors. Mechanical means are the preferred means for effecting passage. For instance, a fine glass needle may be used to cut an ES cell colony from the feeder layer into smaller cell clusters. Repeated pipetting may further break down these clusters. Because of the apparently non-degradative nature of this method, the cells may be passaged at higher dilutions such as 1:100 rather than 1:5 or 1:10. Also, such cells tend to become reestablished more rapidly than cells passaged by chemical or enzymatic methods. The subject ES cells may be passaged indefinitely using the described methodology to create an essentially unlimited supply of undifferentiated ES cells.

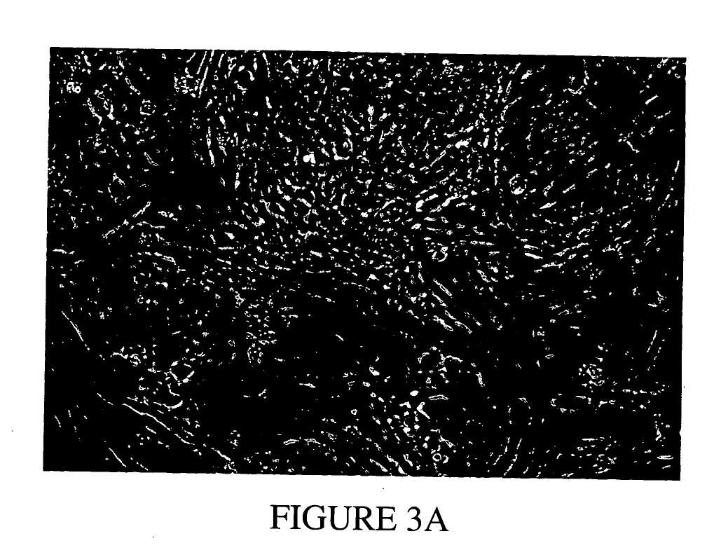

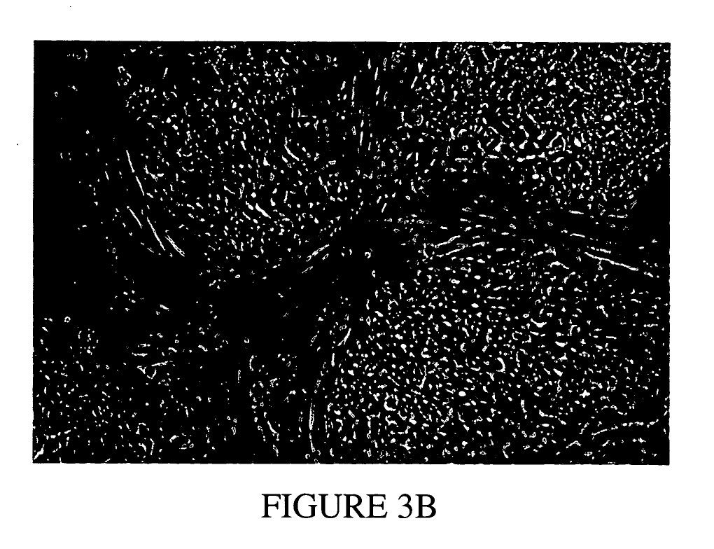

As previously discussed, the morula derived cells used to isolate the subject ES cell lines are morphologically similar to blastocyst initiated stem cells, with the doubling time in the range of about 32-45 hours. The morphology of the ES cell line generated from morula cells compared to those derived from blastocyst, is illustrated by a comparison of FIG. 3A to FIG. 3B. FIG. 3A illustrates the consistent uniformity of the ES cell line derived from the morula cells, as compared to the ES cell line derived from the blastocyst in FIG. 3B.

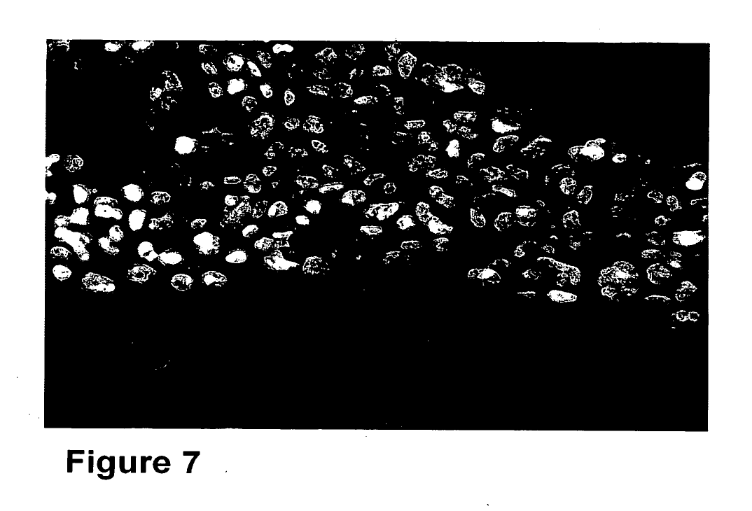

Isolated human ES cells can be positive for the expression of various embryonic stem cell markers, including alkaline phosphatase, SSEA-1, SSEA-3, SSEA4, TRA-1-60, TRA-1-80, TRA-2-39 and Oct-4 (See Table 1). Specifically, in one embodiment of the present invention, the isolated human ES cells are positive for the expression of both Tra-2-39 and Oct-4 (FIG. 7). Tra-2-39 is a marker for L-alkaline phosphatase a specific human liver alkaline phosphatase enzyme [orthophosphoric monoester phosphohydrolase (alkaline optimum), Reference #E.C. 3.1.3.1]., located in the cytoplasm of the cell, while Oct-4 can be detected in the nuclei of morula and ICM derived ES cells. Detection of Oct-4 has been performed with polyclonal antibodies and additional evidences of the presence of Oct-4 have been obtained using RT-PCR products for detecting mRNA Oct-4 having approximately 73 base pairs. The greater level of Oct 4 gene markers present in the cells derived from morula cells, indicate that the ES cell line is more pluripotent.

In further embodiments, it is anticipated that the stem cells will provide materials that may be used for the production of transgenic or genetically altered ES cells, which in turn may be used to produce transgenic or genetically altered derivations of embryonic stem cells. For example, methods for introducing polynucleotides, i.e., desired DNA and/or RNAs, into cells in culture are well known in the art. Such methods include, but are not limited to: electroporation, retroviral vector infection, particle acceleration, transfection, and microinjection. Cells containing the desired polynucleotide (homologous or heterologous to host cell) will be selected according to known methods. The individual cells from a culture of transgenic somatic cells may be used as nuclear transfer donors, a particularly advantageous use of the present invention for certain needs cell therapy. Further, the transgenic or non transgenic morula derived ES cell will facilitate the production of a variety of differentiated cells, having an identical genetic type of major histocompatibility complex (MHC) modification in case when morula taken for establishing embryonic stem cells will be used from nuclear transfer embryo. The derivation of these cell lines may be used for cell therapy.

The present invention will now be further described by the following examples which are provided solely for purposes of illustration and are not intended to be in any way limiting.

### EXAMPLES

### Example 1

### Isolating Morula Derived ES-Cell Lines Using a Human Derived Feeder Layer

Morula stage human embryos were obtained from in-vitro fertilizations. The embryos ranged in size from 8-24 cells and selected between 34 days from the time of fertilization. The procedure is as follows. First, 3 μg/ml of pronase was used to treat the embryos in order to remove the zona pellucida. Morula stage embryos were then placed in HTF-HEPES with 10% Plasmanate. Second, morula stage cells ranging in size from 8-24 cells were placed underneath human skin primary fibroblasts. The primary culture of human skin fibroblasts was obtained from a skin biopsy.

The following was the procedure to develop human skin fibroblasts. The skin biopsy was sliced into 1 mm pieces and placed under a slide cover glass to provide better skin to surface contact with the plastic in the dish. The dish was filled with MEM-Alpha medium. Within several days, human skin fibroblasts were ready to be passaged. To disaggregate cells for passage, a 0.02% EDTA solution was used. Loose cell clusters were then cultured in Petri dishes containing MEM-Alpha supplemented with penicillin, streptomycin, 10% fetal calf serum (FCS) and 0.1 mM 2-mercaptoethanol. Finally, the cells were cultured over a 2-3 week period at 37° C., 5% C02 and 100% humidity. Prior to their usage as feeder cells, they were treated with mitomycin C at 10 μg/ml within 3 hrs and thoroughly washed. The mitomycin C-pretreated fibroblast layer was then used as a feeder cell layer for the blastomeres.

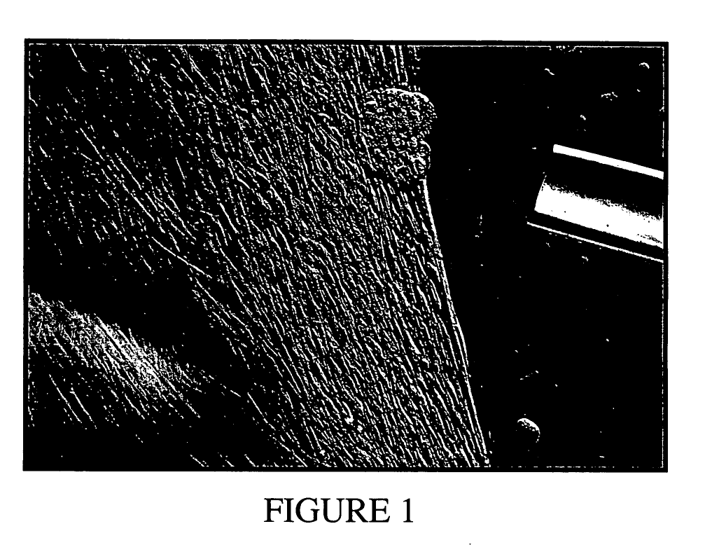

In one experiment the individual blastomeres were placed on top of the feeder cell layer. However, ES cell lines were more readily established and differentiation better inhibited when the blastomeres were placed beneath the feeder layer. (FIG. 1) It is theorized that placing the blastomeres underneath the feeder layer enhances cell-to-cell contact between the morula stage blastomeres and the membrane associated with differentiating inhibiting factors such as LIF and somatomedin proteins that promote development of stem cells. Morula placed on top of the feeder layer had relatively less cell-to-cell contact, and occasionally differentiated into trophoblast vesicles or blastocyst. Every 2-3 days, the MEM-Alpha plus 10% FCS growth medium was replaced. Once the cells had been cultured for a total of approximately 7-10 days, embryonic stem cell multilayer was obtained. Around this time, the blastomeres started to differentiate, exhibiting multilayer appearance.

The multilayer of embryonic stem cells was then passaged onto new mitotically inactive feeder layers. First, disaggregation was accomplished in the presence of EDTA, and mechanically using a fine glass needle micropipette. The needle helped to cut the ES cell multilayer into smaller cell clusters. Split cell clusters were transferred onto fresh mitotically inactivated human fibroblast feeder layers. Specific morphology cell selection of fastest proliferating cells with small amount of cytoplast is required for establishing stem cells. Within two or three initial passages, morula-derived cells emitted different types of cells, including epithelium-, neuron- and fibroblast-like cells.

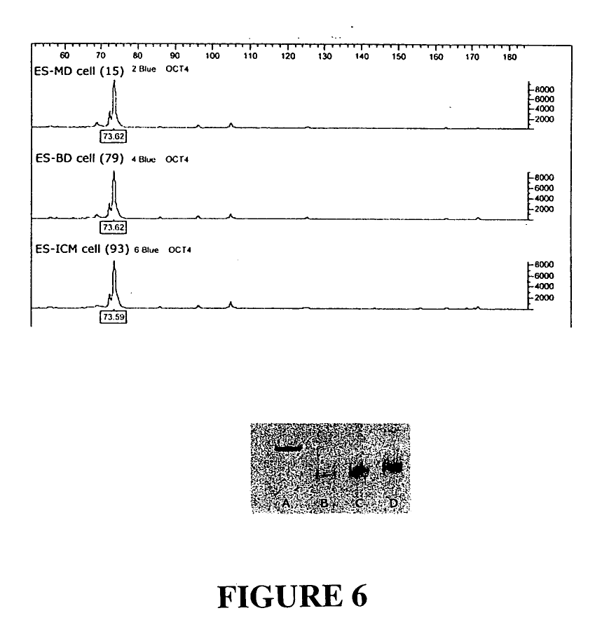

This method resulted in the generation of several ES-cell lines from morula-derived embryos in the 8-24-cell stage, and provided for both male and female ES cell lines. Morula derived cells lines are similar to blastocyst-ICM derived stem cells because they have similar euploid karyotypes and similar morphologies. A small adjacent ring of cytoplasts surrounding a nucleus with prominent nucleoli characterizes this morphology. Additionally, morula derived cell lines and blastocyst-ICM derived cell lines have tested positive for specific stem cell markers including: alkaline phosphatase, SSEA-3, SSEA-4, TRA-1-60, TRA-1-80, TRA-2-39 and Oct-4 (Table 1). Detection of Oct-4 is performed with polyclonal antibodies and additional evidence of the presence of Oct-4 is obtained with RT-PCR products for detection of mRNA Oct-4 expecting to have approximately seventy-three (73) base pairs for morula, blastocyst and ICM derived human embryonic stem cell lines. (FIG. 6)

TABLE 1
Characterization of ES-cell lines derived from different embryo stages.

| Cell Line | Source | # Pas | AP | SS3 | SS4 | T60 | T81 | T39 | Oct4 | Karyotype |
|---|---|---|---|---|---|---|---|---|---|---|
| 15 | Morula | 17 | 100 | 87 | 95 | 97 | 98 | 100 | 94 | 46, XY |
| 18 | Morula | 16 | 100 | 91 | 96 | 95 | 96 | 100 | 96 | 46, XX |
| 21 | Morula | 14 | 100 | + | + | + | + | 100 | 91 | 46, XX |
| 24 | Morula | 10 | 100 | + | + | + | + | 100 | + | 46, XX |
| 27 | Morula | 15 | 100 | 77 | + | + | + | 100 | + | 46, XX |
| 28 | Morula | 16 | 100 | 69 | 89 | 93 | 91 | 100 | 97 | 46, XY |
| 31 | Morula | 10 | 100 | 72 | + | + | + | 100 | + | 46, XX |
| 33 | Morula | 8 | 100 | 84 | + | + | + | 100 | + | 46, XY |
| 53 | Blast | 6 | 100 | + | + | + | + | 100 | + | 46, XX |
| 60 | Blast | 5 | 100 | 84 | 93 | 97 | 98 | 100 | 94 | 46, XX |
| 62 | Blast | 7 | 100 | + | + | + | + | 100 | + | 46, XY |
| 63 | Blast | 6 | 100 | 76 | 92 | 94 | 92 | 100 | 94 | 46, XY |
| 79 | Blast | 7 | 100 | + | + | + | + | 100 | + | 46, XX |
| 80 | Blast | 24 | 100 | + | + | + | + | 100 | + | 46, XX |
| 81 | Blast | 6 | 100 | + | + | + | + | 100 | + | 46, XY |
| 93 | ICM | 5 | 100 | 76 | 91 | 95 | 99 | 100 | 96 | 46, XY |
| 94 | ICM | 5 | 100 | + | + | + | + | 100 | + | 46, XY |
| 95 | ICM | 4 | 100 | + | + | + | + | 100 | + | 46, XX |
| 96 | ICM | 21 | 100 | + | + | + | + | 100 | + | 46, XX |
| 97 | ICM | 3 | 100 | 83 | 96 | 97 | 98 | 100 | 95 | 46, XY |

A continuous undifferentiated culture of morula derived cell lines was maintained for 6 months. After 6 months, the cell lines were frozen in liquid nitrogen.

### Example 2

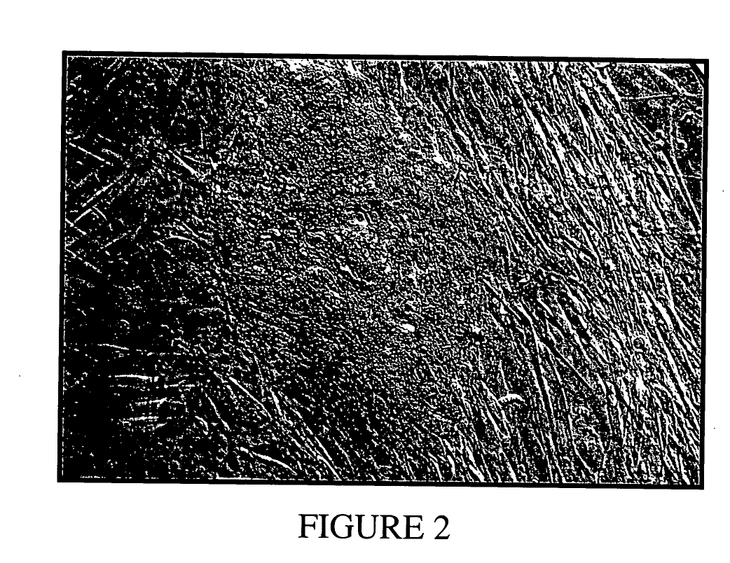

Isolating morula derived ES-cell lines using a mouse derived feeder layer. Morula or compacted morula stage embryos were first isolated using the same manner described above. Morula stage embryos ranging in size from 8-24 cells were placed underneath a mouse fibroblast feeder cell layer prepared according to the method described previously. (FIG. 2) The feeder cell layer was prepared from murine line STO. These cells were treated with mitomycin C at 10 μg/ml for 3.5 hrs and then washed prior to their usage as feeder cells. Every two to three days, the MEM-Alpha plus 10% FCS growth medium was replaced. After the cells had been cultured for a total of about 7-10 days, embryonic stem cell multilayers were obtained. Around this time, the blastomeres started to differentiate, exhibiting embryonic stem cell-like appearance. The cells were then passaged onto new mitotically inactive feeder layers. Passaging was effected mechanically with EDTA and using a fine glass needle micropipette to cut the ES cell multilayer into smaller cell clusters. These cell clusters were then transferred onto fresh mitotically inactivated fibroblast feeder layers. Within two or three initial passages, morula derived cells emitted different types of cells, including epithelium-, neuron- and fibroblast-like cells.

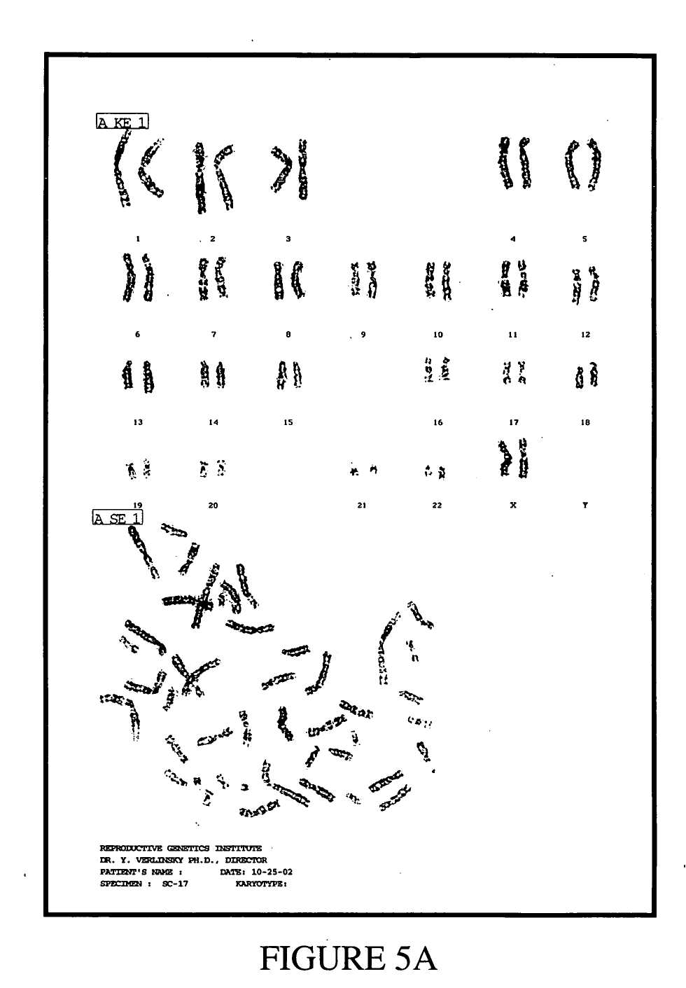

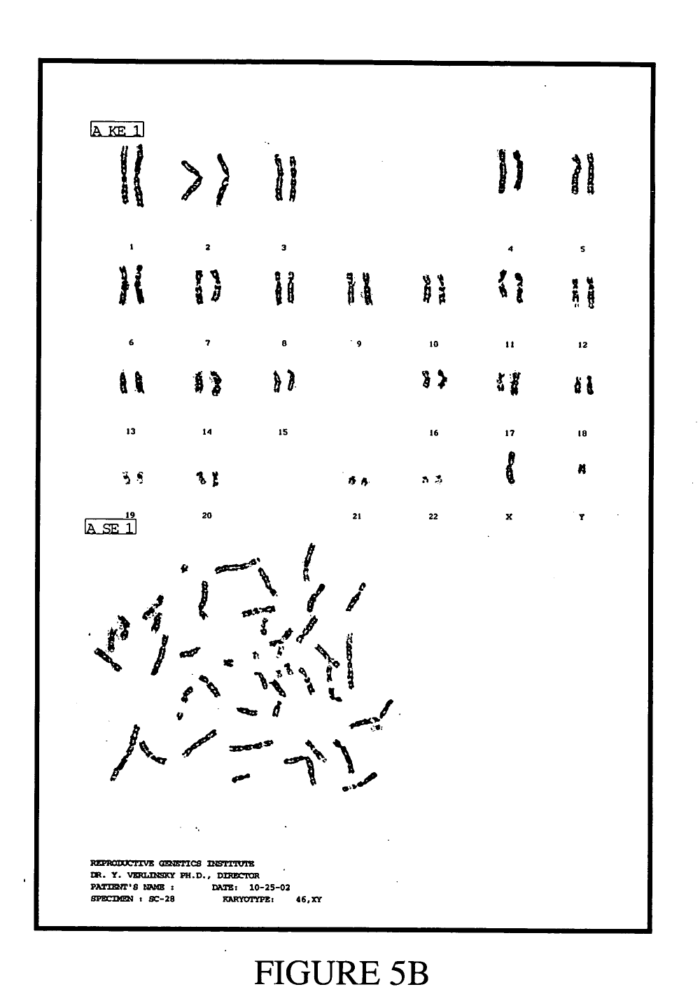

This method resulted in the generation of several ES-cell lines from morula-derived embryos in the 8-24 cell stage. Both male and female ES cell lines were created. (FIGS. 5A and 5B) Morula derived cells lines have euploid karyotypes and are similar in morphology to blastocyst-ICM derived stem cells. A small adjacent ring of cytoplasts surrounding a nucleus with prominent nucleoli characterizes this morphology. Staining morula derived stem cells for alkaline phosphatase with fast blue or fast violet have shown positive clusters of embryonic stem cells. A specific marker for the Oct 4 gene for morula derived embryonic stem cells has been found in lysed embryonic stem cells by RT-PCR. A continuous culture was maintained for 6 months. After 6 months, the cell lines were frozen in liquid nitrogen.

While the specific embodiments have been illustrated and described, numerous modifications come to mind without significantly departing from the spirit of the invention and the scope of protection is only limited by the scope of the accompanying claims.

1. A method for isolating a human pluripotent embryonic stem cell line comprising the steps of:
providing a morula stage human embryo cell;
positioning the morula cells onto a feeder cell layer;
culturing the morula cells to create multiple layers of cells;
passaging the multiple layers of cells onto a second culturing medium for the proliferation of embryonic stem cells.

2. The method of claim 1, wherein the morula cells include blastomeres.

3. The method of claim 1, wherein the morula cells are derived from enucleated oocytes after somatic cell nuclear transfer.

4. The method of claim 1, wherein the step of positioning the morula cells includes positioning in close contact with the feeder cell layer.

5. The method of claim 1, wherein the step of positioning the morula cells includes positioning underneath the feeder cell layer.

6. The method of claim 1, wherein the step of positioning the morula cells includes positioning between a plurality of feeder cell layers.

7. The method of claim 4, wherein the step of positioning the morula cells in close contact with the feeder cell layer prevents differentiation.

8. The method of claim 1, wherein the feeder cell layer is a mitotically inactive feeder layer.

9. The method of claim 1, wherein the step of passaging further includes isolation of individual cells.

10. The method of claim 1, wherein the step of passaging, further includes isolation of a cluster of cells.

11. The method of claim 9, wherein passaging of cells is accomplished by mechanical means.

12. The method of claim 1, wherein step of passaging the cells onto the second culturing medium prevents differentiation of the cells.

13. The method of claim 10, wherein the isolated cells are passaged onto another feeder cell layer.

14. The method of claim 12 wherein the isolated cells can be passaged indefinitely in an undifferentiated state onto a new culture medium creating an unlimited supply of ES cells.

15. The method of claim 1, further comprising the step of selecting embryonic stem cells with relatively low cytoplasm to nucleus ratios.

16. An embryonic stem cell line derived from the method of claim 1.

17. The embryonic stem cell line of claim 16, wherein the cells are positive for TRA-2-39.

18. The embryonic stem cell line of claim 16, wherein the cells are positive for Oct-4.

19. The embryonic stem cell line of claim 16, wherein the cells are positive for Oct-4 and TRA-2-39.

20. A method for isolating a human pluripotent embryonic stem cell line comprising the steps of:
providing a morula stage human embryo cell;
removing a zona pellucida from a morula stage human embryo cell releasing a plurality of blastomeres;
positioning the blastomeres in close contact with a feeder cell layer;
culturing the blastomeres to create multiple layers of cells; and
passaging the multiple layers of cells onto a second culturing medium, wherein the second culturing medium enables further proliferation of cells and prevents differentiation of the resulting cells.

21. The method of claim 20, wherein the step of positioning the blastomeres includes positioning the blastomeres underneath the feeder cell layer.

22. The method of claim 20, wherein the step of positioning the blastomeres includes positioning the blastomeres between a plurality of feeder cell layers.

23. The method of claim 20, wherein the isolated cells are passaged onto another feeder cell layer.

24. An embryonic stem cell line derived from the method of claim 20.

25. The embryonic stem cell line of claim 24, wherein the cells are positive for TRA-2-39.

26. The embryonic stem cell line of claim 24, wherein the cells are positive for Oct-4.

27. The embryonic stem cell line of claim 24, wherein the cells are positive for Oct-4 and TRA-2-39.
